(19) United States
(12) Patent Application Publication
(10) Pub. No.: US 2003/0119159 A1
(43) Pub. Date: Jun. 26, 2003

(54) Aptamer capable of specifically adsorbing to Verotoxin-1 and method for obtaining the aptamer

(75) Inventors: Keisaku Okada, Ibaraki-shi (JP); Shuji Senda, Ibaraki-shi (JP); Akio Kobayashi, Toyonaki-shi (JP); Eiichiro Fukusaki, Suita-shi (JP); Takeshi Honda, Ibaraki-shi (JP); Itaru Yanagihara, Sakai-shi (JP); Tsuyoshi Nakanishi, Yao-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Nitto Denko Corporation, Ibaraki-shi (JP)

(21) Appl. No.: 10/189,916

(22) Filed: Jul. 3, 2002

(30) Foreign Application Priority Data
Jul. 4, 2001 (JP) 203856/2001

(51) Int. Cl. C12N 9/99; C07H 21/04; C12P 21/02; C12N 5/06

(52) U.S. Cl. 435/184; 435/69.2; 435/320.1; 435/325; 536/23.2

(57) ABSTRACT

The present invention provides a method for obtaining an aptamer capable of specifically adsorbing to Verotoxin-1 produced by Enterohemorrhagic Escherichia coli by an in vitro selection method utilizing affinity chromatography, particularly, a method including use of an affinity column immobilizing thereon a peptide having an amino acid sequence of Verotoxin-1, for the affinity chromatography, and a single strand nucleic acid molecule, which is an aptamer capable of specifically adsorbing to Verotoxin-1.

### TECHNICAL FIELD OF THE INVENTION

The present invention relates to a single strand nucleic acid molecule (aptamer) capable of specifically adsorbing to Verotoxin-1 and a method for obtaining the aptamer.

### BACKGROUND OF THE INVENTION

Verotoxins are produced by Enterohemorrhagic Escherichia coli, and classified into type I and type II based on their different amino acid sequences. These toxins consist of one subunit A and five subunits B. The subunit B is known to recognize and bind with the receptor present on an intestinal epithelium cell, and the subunit A is known to bind with rRNA in the cell to inhibit protein synthesis. Verotoxin-1 and Verotoxin-2 have highly similar biological properties, but different physiochemical and immunological properties. Verotoxin-1 is the same toxin as the Shiga toxin produced by Shigella dysenterae type 1, and is called the Shiga-like toxin (SLT) and the like. A high affinity ligand capable of selectively recognizing Verotoxin-1 can be advantageously used for detection and quantitative determination of Verotoxin-1, further for neutralization, trapping and the like of Verotoxin-1, and the like, and therefore, the development of such ligand is of great significance.

A conventional method for obtaining a ligand having a high affinity includes production of an antibody based on the high specificity of antigen-antibody reaction. For production of an antibody against toxin, however, a conventional method includes immunization after detoxication by formaldehyde treatment and heat treatment, to avoid toxicity to the animal to be sensitized. In this case, however, the above-mentioned treatment causes structural changes, making it difficult to obtain a ligand having a high affinity.

### SUMMARY OF THE INVENTION

The present invention aims at solving the above-mentioned problem and provides a method for obtaining a novel affinity ligand capable of specifically recognizing and adsorbing to Verotoxin-I produced by Enterohemorrhagic Escherichia coli, as a target molecule, and the affinity ligand.

As a result of the intensive studies by the present inventors, it has been found that the above-mentioned affinity ligand can be obtained by utilizing a technique developed along with the advance in the evolutionary molecular engineering in recent years, such as an in vitro selection method, SELEX (Systematic evolution of ligands by exponential enrichment) and the like, which involves screening of a nucleic acid molecule, namely, an aptamer, having high affinity for a target molecule (e.g., protein etc.) from a random oligonucleotide library.

Accordingly, the present invention provides the following.

(1) A method for obtaining an aptamer capable of specifically adsorbing to Verotoxin-1 by an in vitro selection method utilizing affinity chromatography, which comprises using a carrier immobilizing thereon a peptide having an amino acid sequence of Verotoxin-1 for the above-mentioned affinity chromatography.

(2) The method of the above-mentioned (1), wherein the peptide having an amino acid sequence of Verotoxin-1 has an amino acid sequence consisting of 3 to 30 amino acids from the amino acid sequence of Verotoxin-1.

(3) The method of the above-mentioned (1), wherein the carrier is packed in an affinity column.

(4) The method of the above-mentioned (1), wherein the peptide has an amino acid sequence depicted in SEQ ID NO: 1.

(5) The method of the above-mentioned (1), wherein the peptide has 6 histidines at an N-terminal or C-terminal and is immobilized on the carrier via said 6 histidines.

(6) A single strand nucleic acid molecule which is an aptamer obtained by the method of the above-mentioned (1).

(7) A method for obtaining an aptamer capable of specifically adsorbing to Verotoxin-1 by an in vitro selection method utilizing affinity chromatography, which comprises using an affinity column immobilizing thereon a peptide having an amino acid sequence consisting of 3 to 30 amino acids from the amino acid sequence of Verotoxin-1.

(8) The method of the above-mentioned (7), wherein the above-mentioned peptide has an amino acid sequence depicted in SEQ ID NO: 1.

(9) The method of the above-mentioned (7) or (8), wherein the above-mentioned peptide has 6 histidines at an N-terminal or C-terminal and is immobilized on the affinity column via said 6 histidines.

(10) A single strand nucleic acid molecule, which is an aptamer obtained by any of the above-mentioned (7)-(9).

(11) A single strand nucleic acid molecule, which is an aptamer capable of specifically adsorbing to Verotoxin-1 and which has a base sequence of any of the following (a) to (e):

(a) a base sequence consisting of 38^{th}-96^{th} nucleotides depicted in SEQ ID NO: 3, provided that when the nucleic acid molecule is an RNA, T in the sequence is U,

(b) a base sequence consisting of 38^{th}-96^{th} nucleotides depicted in SEQ ID NO: 4, provided that when the nucleic acid molecule is an RNA, T in the sequence is U,

(c) a base sequence consisting of 40^{th}-88^{th} nucleotides depicted in SEQ ID NO: 5, provided that when the nucleic acid molecule is an RNA, T in the sequence is U,

(d) a base sequence consisting of 39^{th}-82^{nd} nucleotides depicted in SEQ ID NO: 6, provided that when the nucleic acid molecule is an RNA, T in the sequence is U,

(e) any of the above-mentioned base sequences (a) to (d), wherein 1 to several nucleotides have been deleted, substituted, inserted or added.

(12) The single strand nucleic acid molecule of the above-mentioned (11), wherein the nucleic acid is a DNA.

### BRIEF DESCRIPTION OF THE DRAWING

FIG. 1 is a flow chart showing a series of steps of a preferable method of the present invention for obtaining an aptamer capable of specifically adsorbing to Verotoxin-1.

### DETAILED DESCRIPTION OF THE INVENTION

In the present specification, by the “in vitro selection method” is meant a method for obtaining a nucleic acid molecule having a particular function by repeatedly performing a selection process including separation of a single strand oligonucleotide having a particular function (e.g., specific adsorption to a target substance) from a randomly synthesized single strand oligonucleotide library, amplification of the oligonucleotide, and separation of a single strand oligonucleotide having the above-mentioned particular function. When a nucleic acid molecule (aptamer) capable of specific adsorption to a particular target substance is to be obtained from a random oligonucleotide library, the above-mentioned nucleic acid molecule capable of adsorption is separated by, for example, affinity chromatography using an affinity column having a target substance immobilized thereon.

In the present specification, by the “aptamer” is meant a single strand nucleic acid molecule capable of specific adsorption to a particular target substance. The aptamer in the present specification is not limited to those obtained by the above-mentioned in vitro selection method.

In the present specification, by the “affinity chromatography” is meant a separation method utilizing a specific interaction (affinity) that a biological substance shows. The separation means is not particularly limited, and various methods usually employed in the pertinent field are used. To be specific, affinity chromatography using an affinity column is exemplified. This method includes at least the steps of (i) applying a substance capable of specifically adsorbing to a target substance and/or a substance incapable of adsorbing to an affinity column packed with a carrier having a target substance immobilized thereon (hereinafter sometimes to be conveniently referred to as a target substance-immobilized affinity column), (ii) washing, after the application, the column with a washing buffer to separate the above-mentioned substance capable of adsorption from a substance incapable of adsorption (washing treatment), and (iii) weakening, after the washing treatment, the bonding force between a substance capable of adsorption and the target substance immobilized on the column, with an elution buffer to allow elution of the substance capable of adsorption (elution treatment). As the carrier used for immobilizing the target substance, those known to be used for affinity chromatography, particularly affinity column chromatography, are mentioned.

### EMBODIMENT OF THE INVENTION

The present invention is described in detail in the following.

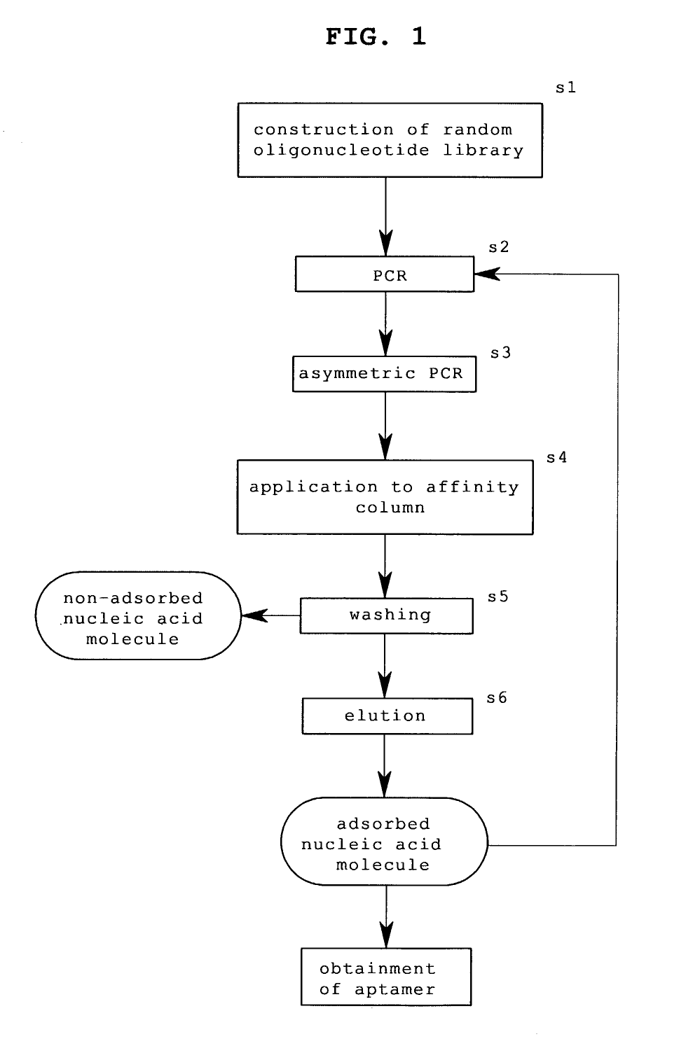

FIG. 1 is a flow chart showing a series of steps of a preferable method of the present invention for obtaining an aptamer capable of specifically adsorbing to Verotoxin-1.

In Step s1, a library of a single strand oligonucleotide (hereinafter sometimes to be also referred to as ssNt) containing a random region of a predetermined length of about 30 base-about 80 base is prepared using an automatic DNA/RNA synthesizer according to a conventional method. This library preferably contains 10¹³-10¹⁴ or more kinds of ssNt.

To facilitate PCR amplification in Step s2, s3 to be mentioned below, each ssNt is preferably designed to have a common priming site on both ends of the random region (i.e., a sequence homologous with sense primer on 5′-terminal and a sequence complementary to anti-sense primer on 3′-terminal), wherein “sense” and “anti-sense” primers are used to amplify the original ssNt and complementary chain thereof. Each of such priming sites is preferably designed to have a length of about 15 base-about 40 base, preferably about 15 base-about 30 base, and the corresponding PCR primers meet the general requirements of preferable primers.

When the aptamer after selection needs to be subcloned to a suitable vector, the priming site may contain a suitable restriction enzyme recognition site to facilitate the cloning. However, when amplification is performed by asymmetrical PCR such that a single strand oligonucleotide occupies the majority of the resulting amplified products, as in the embodiment shown in FIG. 1, the aptamer can be directly sequenced without subcloning.

In the subsequent Step s2, double stranded oligonucleotide (hereinafter sometimes to be referred to as dsNt) is amplified with the obtained ssNt library as a template and using sense and anti-sense primers corresponding to the priming sites on both ends of ssNt. Amplification of this dsNt can be performed by PCR according to a conventional method.

In the embodiment shown in FIG. 1, the above-mentioned dsNt library amplified by PCR is subjected to asymmetrical PCR using a sense primer alone in Step s3 to follow, whereby ssNt pool wherein sense strand (i.e., original ssNt) alone is amplified is prepared. This is because an aptamer is considered to have a specific adsorption capability to a target substance based on its structural and sequence characteristics that it is a single strand nucleic acid molecule capable of forming a specific secondary structure, and therefore, it needs to be a single strand having an established given secondary structure before affinity chromatography in Steps s4-s6 below.

The ssNt amplified by asymmetrical PCR can be purified by agarose or polyacrylamide gel electrophoresis. Where desired, a PCR product may be subjected to ethanol precipitation for concentration prior to electrophoresis. A gel portion containing a band corresponding to the desired ssNt is recovered and ssNt is purified by a conventional method. ssNt is denatured at not lower than 90° C. prior to affinity chromatography and allowed to cool to ambient temperature to form a suitable secondary structure.

In the present invention, moreover, PCR may be performed by adding a sense primer in a great excess relative to the anti-sense primer (e.g., about 50-100:1), instead of PCR in the above-mentioned Step s2 and asymmetrical PCR in the above-mentioned Step s3, to prepare an ssNt pool wherein only sense strand is amplified.

The subsequent Step s4, Step s5 and Step s6 constitute a series of treatments of affinity chromatography using an affinity column in which a particular peptide is immobilized. What is significant in the present invention is the use of a peptide having an amino acid sequence of Verotoxin-1, particularly peptide having an amino acid sequence consisting of 3 to 30 amino acids, preferably 5 to 25 amino acids, selected from the amino acid sequence of Verotoxin-1, as peptide to be immobilized on this affinity column. This peptide is preferably present in the outer side in a higher order structure, affording recognition of aptamer. Verotoxin-1 consists of one subunit A and five subunits B. Both the subunit A and subunit B have the amino acid sequences known in the pertinent field (subunit A consisting of 293 amino acids and subunit B consisting of 69 amino acids). In the present invention, this peptide having an amino acid sequence of Verotoxin-1 is immobilized on a carrier. In view of the size and strong affinity thereof, this peptide is preferably a partial amino acid sequence of Verotoxin-1, and more preferably an amino acid sequence having 3 to 30 amino acids, and the peptide containing the amino acid sequence is immobilized on an affinity column. The peptide to be used in the present invention can be synthesized using an automatic peptide synthesizer as generally done by those of ordinary skill in the art. It may be naturally occurring or semi-artificial, which can be obtained by an optional method. When the number of amino acids in the above-mentioned amino acid sequence derived from Verotoxin-1 is less than 3, the affinity to select aptamer by affinity chromatography cannot be sufficiently performed. In addition, when the number of amino acids in the amino acid sequence derived from Verotoxin-1 exceeds 30, peptide synthesis becomes difficult, and non-specific adsorption increases, making selection of the aptamer difficult. The amino acid sequence of the peptide may be an amino acid sequence derived from either subunit A or subunit B of Verotoxin-1.

The peptide to be used in the present invention need only be derived from the above-mentioned Verotoxin-1 and not inhibit presentation of affinity. For example, an amino acid sequence containing 3 to 30 amino acids derived from Verotoxin-1 (hereinafter sometimes to be referred to as a high affinity region) may contain amino acid residue(s) at the N-terminal and/or C-terminal, as long as presentation of the affinity is not inhibited. From the aspect of affinity by the above-mentioned high affinity region, the total number of amino acids of peptide is, whether or not the above-mentioned amino acid residue is added, preferably 3 to 30 amino acids, more preferably 5 to 25 amino acids.

The high affinity region of the peptide to be used in the present invention preferably has the following amino acid sequence:

N-Leu-Ser-Ala-Gln-Ile-Thr-Gly-Met-Thr-Val-Thr-Ile-Lys-Thr-Asn-Ala-Cys-His-C (SEQ ID NO: 1)

The amino acid sequence of the above-mentioned SEQ ID NO: 1 is a partial amino acid sequence of subunit B of Verotoxin-1, which is located on the outer side of Verotoxin-1 having a higher order structure and considered to be the region presenting antigenicity.

In the present invention, the above-mentioned peptide preferably has 6 histidines (histidine tag) at the N-terminal or C-terminal, and is preferably immobilized via the histidine tag. In other words, in the amino acid residue other than the high affinity region of peptide, when the amino acid residue is present at the N-terminal of the above-mentioned amino acid sequence region, the N-terminal has a histidine tag, when the amino acid the residue is present at the C-terminal of the above-mentioned amino acid sequence region, the C-terminal has a histidine tag, and when the amino acid residue is present at both terminals of the above-mentioned amino acid sequence regions, the N-terminal or C-terminal has a histidine tag. The peptide is preferably immobilized on the affinity column via the histidine tag. By the action of the above-mentioned histidine tag, the imidazole group of histidine can firmly binds to a nickel ion complex or cobalt ion complex, and by the addition of imidazole or decrease in pH, elution of the affinity ligand is facilitated. As a result, stable affinity chromatography that affords easy control can be realized by affinity column using a support stably retaining a nickel ion complex or cobalt ion complex. As the above-mentioned nickel ion complex or cobalt ion complex, those conventionally known can be used. The peptide to be used in the present invention preferably has a sequence (SEQ ID NO: 2) consisting of the amino acid sequence depicted in the above-mentioned SEQ ID NO: 1 and 6 histidines added to the N-terminal thereof, which it is immobilized on the affinity column upon bonding with a nickel ion complex via the histidine tag.

In the present invention, an affinity column packed with beads and gel solid phase, on which the above-mentioned peptide have been immobilized, is used for the affinity column chromatography of Steps s4-s6. As the column packing material, silica gel and sepharose gel are exemplified.

As mentioned above, Step s4, Step s5 and Step s6 are directed to a series of treatments using affinity chromatography using an affinity column immobilizing the above-mentioned peptide. To be specific, in Step s4, ssNt having a suitable secondary structure and obtained in Step s3 is applied to affinity column immobilizing the above-mentioned peptide, in Step s5, the affinity column is washed and the nucleic acid molecule that failed to adsorb to the above-mentioned peptide (hereinafter sometimes to be referred to as non-adsorbed nucleic acid molecule) is separated (washing treatment), and in Step s6, nucleic acid molecule that specifically adsorbed to the above-mentioned peptide (hereinafter sometimes to be referred to as adsorbed nucleic acid molecule) is eluted from the affinity column (elution treatment). According to the in vitro selection method of the present invention, adsorbed nucleic acid molecule is separated from non-adsorbed nucleic acid molecule utilizing the affinity chromatography using an affinity column immobilizing the above-mentioned peptide. The washing buffer to be used for the above-mentioned washing treatment, and the elution buffer to be used for the above-mentioned elution treatment may be those conventionally and widely used in the pertinent fields. The elution buffer may be a washing buffer containing imidazole.

The adsorbed nucleic acid molecule obtained by the above-mentioned Step s4-s6 is subjected to at least 5, preferably about 7-15, cycles of the above-mentioned PCR amplification (Step s2), asymmetrical PCR (Step s3) and affinity chromatography (Step s4-s6) using an affinity column immobilizing the above-mentioned peptide, whereby the aptamer of the present invention can be obtained.

The obtained aptamer is made to be double stranded according to a conventional method and subcloned to a suitable vector using the restriction enzyme recognition site constructed in the priming site, by blunting the end, or by TA cloning method, after which its base sequence can be determined by the Maxam-Gilbert method or dideoxy method. Alternatively, the obtained single strand aptamer can be directly sequenced without subcloning.

According to the above-mentioned method of the present invention, an affinity ligand capable of specifically recognizing and adsorbing to Verotoxin-1 as a target substance, which has been conventionally difficult to obtain, can be efficiently obtained.

The aptamer of the present invention capable of specifically adsorbing to Verotoxin-1 is a single strand DNA or RNA, preferably a single strand DNA. While the length thereof is not particularly limited, it is preferably about 30 base-about 120 base.

In the preferable embodiment, the aptamer of the present invention substantially comprises a base sequence selected from the group consisting of a base sequence consisting of 38^{th} to 96^{th} nucleotides of the base sequence depicted in SEQ ID NO: 3, a base sequence consisting of 38^{th} to 96^{th} nucleotides of the base sequence depicted in SEQ ID NO: 4, a base sequence consisting of 40^{th} to 88^{th} nucleotides of the base sequence depicted in SEQ ID NO: 5, and a base sequence consisting of 39^{th} to 82^{nd} nucleotides of the base sequence depicted in SEQ ID NO: 6, wherein, when the nucleic acid molecule is RNA, T in the sequence is U. As used herein, by the “substantially comprises” is meant that any of the above-mentioned base sequences per se is included or any of the above-mentioned base sequences, wherein 1 or several nucleotides have been deleted, substituted, inserted or added and the Verotoxin-1 specific adsorption capability is retained, is included.

A single strand nucleic acid molecule (aptamer) substantially comprising the above-mentioned base sequence may not be prepared by the aforementioned method of the present invention, but may be prepared by any method, though preference is given to one prepared by the aforementioned method of the present invention.

### EXAMPLES

The present invention is explained in detail by referring to Examples. The Examples are mere exemplifications and do not limit the present invention in any way.

### Example 1

[1] Preparation of Amplified Single Strand DNA (ssDNA) Library

(1) Using an automatic DNA synthesizer, the following template DNA with 59 mer as a random region and a sense (P1) primer and an anti-sense (P2) primer were synthesized (Step s1).

Reaction Conditions

| | |
|---|---|
| initial denaturation | 94° C., 1 min |
| denaturation | 94° C., 15 sec |
| annealing | 55° C., 15 sec 10 cycles |
| extension | 72° C., 15 sec |
| final extension | 72° C., 6 min |

(3) Using the above-mentioned PCR product as a template and P1 alone as a primer, asymmetrical PCR was performed (Step s3) to ultimately prepare 2 ml of PCR product (100 μl×20 tubes). The reaction mixture composition and reaction conditions were as follows.

Reaction Mixture Composition

| | | |
|---|---|---|
| distilled water | 78.5 | μl |
| 10 × PCR buffer | 10 | μl |
| 20 mM dNTPs | 1 | μl |
| 10 μM P1 primer | 5 | μl |
| 1 μg/ml template DNA | 5 | μl |
| Ex Taq ™ DNA polymerase | 0.5 | μl (2.5 units) |

Reaction Conditions

| | |
|---|---|
| initial denaturation | 94° C., 1 min |
| denaturation | 94° C., 15 sec |
| annealing | 55° C., 15 sec 40 cycles |
| extension | 72° C., 15 sec |
| final extension | 72° C., 6 min |

The PCR reaction mixture was dispensed by 400 μl to 5 microtubes. Thereto were added 10M ammonium acetate (80 μl) and 99.5% ethanol (1 ml) and gently mixed. The mixture was stood at −80° C. for 20 min. The mixture was centrifuged at 15,000 rpm for 15 min, rinsed with 70% ethanol and centrifuged at 15,000 rpm for 10 min. The precipitate was vacuum dried. Sterile distilled water (20 μl) was added and the mixture was vigorously mixed on Voltex to solve the precipitate. A gel loading buffer (20 μl, 95% formamide, 0.5 mM EDTA (pH 8.0), 0.025% STS, 0.025% xylene cyanol, 0.025% Bromophenol blue) was added and the mixture was thoroughly mixed on Voltex. The mixture was treated at 90° C. for 3 min to allow denaturation. The mixture was rapidly cooled on ice and subjected to electrophoresis (150 V, 50 min) on polyacrylamide gel. After immersing in ethidium bromide solution for about 5 min, the gel was washed with water and detected for a band on a transilluminator. The gel portion containing the objective band was cut out and ruptured. Elution buffer (800 μl, 0.5M ammonium acetate, 10 mM magnesium acetate, 1 mM EDTA (pH 8.0), 0.1% STS) was added and the mixture was shaken for 3 hr, and passed through a filter to recover the filtrate.

[2] Affinity Column Chromatography

(1) The DNA obtained in the above-mentioned [1] was precipitated with ethanol and, after vacuum drying, dissolved in distilled water (100 μl). A 2×binding buffer (100 μl, 200 mM Tris-HCl, 400 mM NaCl, 50 mM KCl, 20 mM MgCl₂ (pH 8.0)) was added and thoroughly mixed and absorbance at 260 nm was measured. The DNA solution was treated at 90° C. for 5 min to allow denaturation, allowed to cool naturally and held. Formation of the secondary structure was confirmed by changes in absorbance.

(2) A peptide in Verotoxin-1 subunit B was synthesized with a automatic peptide synthesizer. 6 Histidines were added to the N-terminal for synthesis of the peptide having the following amino acid sequence:

N-His-His-His-His-His-His-Leu-Ser-Ala-Gln-Ile-Thr-Gly-Met-Thr-Val-Thr-Ile-Lys-Thr-Asn-Ala-Cys-His-C (SEQ ID NO: 2)

(3) The above-mentioned peptide was immobilized on a column material as follows. The above-mentioned peptide (10 μg) was dissolved in 1×binding buffer (800 μl, 100 mM Tris-HCl, 200 mM NaCl, 25 mM KCl, 10 mM MgCl₂ (pH 8.0)) and NTA-Ni²⁺ agarose gel (200 μl) was added. The mixture was shaken at room temperature for 1 hr. The immobilized gel was packed in a 8 mm×5 mm column, and equilibrated by washing with an about 20-fold amount of 1×binding buffer, wherein the solution obtained then was used as a baseline.

(4) The DNA sample obtained in the above-mentioned (1) was applied to a column, and the eluate was received in a microtube upon opening the cock and applied again to the column (Step s4). This operation was repeated 3 times, and the column was left standing at room temperature for 30 min. A 1×binding buffer (5 ml) was poured and the cock was opened to fractionate in 6 microtubes by about 12 drops (about 650 μl) (Step s5). The cock was closed once, an elution buffer (100 mM Tris-HCl, 200 mM NaCl, 25 mM KCl, 10 mM MgCl₂ (pH 8.0)+250 mM imidazole) was poured thereon and the cock was opened. The eluate was received in a microtube and returned again to the column. This operation was repeated 3 times, whereby the buffer was substituted by an elution buffer. The cock was opened again to fractionate in 3 microtubes by about 12 drops (Step s6). The eluate was divided by 400 μl and glycogen (2 μl) was added thereto, followed by ethanol precipitation and vacuum drying. The precipitate was thoroughly dissolved in water (15 μl).

[3] Identification of Verotoxin-1 Specific DNA Aptamer

Each operation (Step s2-s6) of the above-mentioned [1](2)-[2](4) was repeated 12 times. The base sequence of 5 kinds of Verotoxin-1 specific single strand DNA aptamers selected by the operation was determined by the dideoxy method.

The determined base sequence of random regions of each clones was as follows.

What is claimed is

1. A method for obtaining an aptamer capable of specifically adsorbing to Verotoxin-1 by an in vitro selection method utilizing affinity chromatography, which comprises using a carrier immobilizing thereon a peptide having an amino acid sequence of Verotoxin-1 for the affinity chromatography.

2. The method of claim 1, wherein the peptide having an amino acid sequence of Verotoxin-1 has an amino acid sequence consisting of 3 to 30 amino acids from the amino acid sequence of Verotoxin-1.

3. The method of claim 1, wherein the carrier is packed in an affinity column.

4. The method of claim 1, wherein the peptide has an amino acid sequence depicted in SEQ ID NO: 1.

5. The method of claim 1, wherein the peptide has 6 histidines at an N-terminal or C-terminal and is immobilized on the carrier via said 6 histidines.

6. A single strand nucleic acid molecule which is an aptamer obtained by the method of claim 1.

7. A method for obtaining an aptamer capable of specifically adsorbing to Verotoxin-1 by an in vitro selection method utilizing affinity chromatography, which comprises using an affinity column immobilizing thereon a peptide having an amino acid sequence consisting of 3 to 30 amino acids from the amino acid sequence of Verotoxin-1.

8. The method of claim 7, wherein said peptide has an amino acid sequence depicted in SEQ ID NO: 1.

9. The method of claim 7, wherein said peptide has 6 histidines at an N-terminal or C-terminal and is immobilized on the affinity column via said 6 histidines.

10. The method of claim 8, wherein said peptide has 6 histidines at an N-terminal or C-terminal and is immobilized on the affinity column via said 6 histidines.

11. A single strand nucleic acid molecule, which is an aptamer obtained in claim 7.

12. A single strand nucleic acid molecule, which is an aptamer obtained in claim 8.

13. A single strand nucleic acid molecule, which is an aptamer obtained in claim 9.

14. A single strand nucleic acid molecule, which is an aptamer obtained in claim 10.

15. A single strand nucleic acid molecule, which is an aptamer capable of specifically adsorbing to Verotoxin-1 and which has a base sequence of any of the following (a) to (e):
(a) a base sequence consisting of 38^{th}-96^{th} nucleotides depicted in SEQ ID NO: 3, provided that when the nucleic acid molecule is an RNA, T in the sequence is U,
(b) a base sequence consisting of 38^{th}-96^{th} nucleotides depicted in SEQ ID NO: 4, provided that when the nucleic acid molecule is an RNA, T in the sequence is U,
(c) a base sequence consisting of 40^{th}-88^{th} nucleotides depicted in SEQ ID NO: 5, provided that when the nucleic acid molecule is an RNA, T in the sequence is U,
(d) a base sequence consisting of 39^{th}-82^{nd} nucleotides depicted in SEQ ID NO: 6, provided that when the nucleic acid molecule is an RNA, T in the sequence is U,
(e) any of the base sequences (a) to (d), wherein 1 to several nucleotides have been deleted, substituted, inserted or added.

16. The single strand nucleic acid molecule of claim 15, wherein the nucleic acid is a DNA.
